(19) United States
(12) Patent Application Publication
(10) Pub. No.: US 2009/0048617 A1
(43) Pub. Date: Feb. 19, 2009

(54) Implantable mesh for surgical reconstruction in the area of the pelvic floor

(75) Inventor: Bruce Farnsworth, NSW (AU)

Correspondence Address: WENDEROTH, LIND & PONACK, L.L.P., 2033 K STREET N. W., SUITE 800, WASHINGTON, DC, 20006-1021, US

(73) Assignee: A.M.I. AGENCY FOR MEDICAL INNOVATIONS GMBH

(21) Appl. No.: 12/285,769

(22) Filed: Oct. 14, 2008

Related U.S. Application Data
(63) Continuation of application No. PCT/AT2007/000157, filed on Apr. 6, 2007.

(30) Foreign Application Priority Data
Apr. 14, 2006 (AT) A 650/2006

(51) Int. Cl. A61B 17/08; A61F 2/00

(52) U.S. Cl. 606/151; 600/37

(57) ABSTRACT

An implantable mesh for the surgical reconstruction in the area of the pelvic floor includes an anterior mesh segment between the bladder and the vagina, a posterior mesh segment between the vagina and the rectum, a pair of distal transobturator holding straps extending from the anterior mesh segment, a pair of proximal transobturator holding straps extending from the anterior mesh segment and a pair of lower dorsal holding straps extending from the posterior mesh segment. The mesh further includes an intermediate segment located between the anterior mesh segment and the posterior mesh segment. The pair of lower dorsal holding straps extends from the region of the posterior mesh segment bordering on the intermediate segment, and wherein further a pair of upper dorsal holding straps extends from the region of the anterior mesh segment bordering on the intermediate segment.

This is a Continuation of International Application Serial No. PCT/AT2007/000157, filed Apr. 6, 2007, the entire disclosure of which is incorporated herein by reference.

### BACKGROUND OF THE INVENTION

a) Field of the Invention

The invention relates to an implantable mesh for the surgical reconstruction in the area of the pelvic floor, with an anterior mesh segment for the disposition between the bladder and the vagina, a posterior mesh segment for the disposition between the vagina and the rectum, a pair of distal transobturator holding straps for the positioning starting from the anterior mesh segment and extending laterally and ventrally through the obturator, a pair of proximal transobturator holding straps starting from the anterior mesh segment for the positioning starting from the anterior mesh segment and extending laterally and ventrally through the obturator and a pair of lower dorsal holding straps extending from the posterior mesh segment for the positioning starting from the posterior mesh segment and extending laterally and dorsally.

b) Description of Related Prior Art

Implantable meshes for surgical reconstructions in the area of the female pelvic floor are known. An anterior restoration (reconstruction), in which the mesh is implanted in the region between the bladder and the vagina, is carried out in particular in the case of a bladder prolapse (=cystocele). A posterior restoration (reconstruction), in which the mesh is implanted between the vagina and the rectum, is in particular carried out in the case of a rectal prolapse (=rectocele). In a total reconstruction an anterior and a posterior reconstruction are combined, wherein conventionally, after the uterus has been removed, a continuous mesh with an anterior mesh segment and a posterior mesh segment is emplaced.

A known mesh for carrying out an anterior and posterior reconstruction comprises holding straps for securing the mesh on body structures, the straps in each instance extending pairwise from both sides of the mesh. From the anterior mesh segment herein extend two pairs of obturator holding straps. In the implanted state of the mesh, starting from the anterior mesh segment these holding straps extend laterally and ventrally through the transobturator and are brought out through the skin below the pubic bone. During the operation after the mesh has been set in, an appropriate tensile force can be applied onto these sections brought out through the skin in order to properly position the mesh. The sections projecting from the skin are subsequently trimmed before the skin is sutured. The posterior mesh segment furthermore comprises a pair of dorsal holding straps, which, starting from the posterior mesh segment are positioned laterally and dorsally during the surgery and are either sutured to the sacrospinous ligament or are passed through the sacrospinous ligament and deflected in the ventral direction and passed through the transobdurator and the skin in the region below the pubic bone in order to be trimmed after the mesh has been properly aligned.

Through this known implantable mesh for a total reconstruction optimal support of the body structures to be reconstructed is not yet achieved. In particular, there is the risk of a shortening in the region of the proximal vagina and pain and dysparennia can result. Moreover, the posterior support against a rectal prolapse is also not optimal.

If from this conventional mesh parts are cut off such that substantially either only the anterior mesh segment or only the posterior mesh segment remain, a reconstruction that is only an anterior one or a reconstruction that is only a posterior one can be performed with this conventional mesh. In this case the support of the body structures is also not optimal.

### SUMMARY OF THE INVENTION

The invention addresses the problem of providing an implantable mesh of the above described type through which an improved reconstruction is made possible.

According to the invention this is achieved through an implantable mesh for surgical reconstruction in the area of the pelvic floor, comprising

an anterior mesh segment (2) for the disposition between the bladder (4) and the vagina (5),

a posterior mesh segment (3) for the disposition between the vagina (5) and the rectum (10),

an intermediate segment (13) located between the anterior mesh segment (2) and the posterior mesh segment (3),

a pair of distal transobturator holding straps (6) starting from the anterior mesh segment (2) for the positioning starting from the anterior mesh segment (2) and extending laterally and ventrally through the obturator (26),

a pair of proximal transobturator holding straps (7) starting from the anterior mesh segment (2) for the positioning starting from the anterior mesh segment (2) and extending laterally and ventrally through the obturator (26),

a pair of lower dorsal holding straps (15) starting from the posterior mesh segment (3) in the region of the posterior mesh segment (3) bordering on the intermediate segment (13) for the positioning starting from the posterior mesh segment (3) and extending laterally and dorsally, and

a pair of upper dorsal holding straps (14) staring from the anterior mesh segment (2) in the region of the anterior mesh segment (2) bordering on the intermediate segment (13) for the positioning starting from the anterior mesh segment (2) and extending laterally and dorsally.

When carrying out a total reconstruction, a natural reconstruction of the proximal vagina is attained through an implantable mesh according to the invention. The intermediate segment of the mesh lies herein in front of the proximal end of the vagina and through the pairs of dorsal holding straps located on both sides of the intermediate segment and bordering it, good alignment and securement of the mesh can be attained.

The posterior mesh segment is advantageously additionally provided with at least one pair of translevator holding straps. In the implantation of the mesh these straps extending laterally and ventrally can be passed through the levator muscle and subsequently be brought out through the skin, wherein appropriate tensile stress can be applied for adjusting the mesh. After the operation these holding straps can be trimmed beneath the skin, whereupon the skin is sutured. Herein at least one pair of proximal translevator holding straps and one pair of distal translevator holding straps is preferably provided, which are passed through the levator muscle further proximally and further distally.

If within this document the terms “proximal” and “distal” are used, they are in each case understood to be relative to the position of the uterus, i.e. a proximal part is closer to the uterus than a distal part.

In an advantageous embodiment of the invention the intermediate segment of the mesh is provided with bilaterally projecting flaps. During surgery these can be placed in contact on the lateral walls of the proximal vagina in order to attain support against being pushed in by adjacent structures.

### BRIEF DESCRIPTION OF THE DRAWING

Further advantages and details of the invention will be explained in the following in conjunction with the attached drawings, in which:

FIG. 1 is a highly schematic illustration, not to scale, of an implanted mesh according to the prior art for a total surgical reconstruction,

FIG. 2 is an illustration corresponding to FIG. 1 for a mesh according to the invention,

FIG. 3 is an illustration corresponding to FIG. 2, according to a modified installation of the mesh according to the invention,

FIG. 4 is a view of a mesh according to the invention,

FIG. 5 is a highly schematic illustration, not to scale, of the implanted mesh in a section in the region between bladder and vagina, wherein the anterior segment of the mesh is visible,

FIG. 6 is a highly schematic illustration, not to scale, of the implanted mesh in a section in the region between vagina and rectum, wherein the posterior segment of the mesh is visible,

FIG. 7 is an oblique view of a surgical instrument for pulling in a holding strap,

FIG. 8 is a perspective illustration of the end section of the instrument with an end section of the holding strap of the mesh with a connection element for the connection with the instrument,

FIG. 9 is a view of an opposite end section of the instrument with an end section of a coupleable further medical instrument,

FIG. 10 shows a further embodiment of a medical instrument for pulling in a holding strap,

FIG. 11 is a view of an end section of this instrument with an end section of the holding strap of the mesh with an adapted connection element,

FIG. 12 is a view of the opposite end section of the instrument coupled to a further medical instrument,

FIG. 13 is a view of a further embodiment of a medical instrument for pulling in a holding strap,

FIG. 14 is a view of an end section of the instrument and an end section of a holding strap,

FIG. 15 is a view of an opposite end section of the instrument coupled to a further medical instrument,

FIG. 16 is a view of an end section of an instrument for pulling in a holding strap according to a further embodiment variant with an end section of the holding strap which comprises an adapted connection element.

### DETAILED DESCRIPTION OF THE PREFERRED EMBODIMENTS

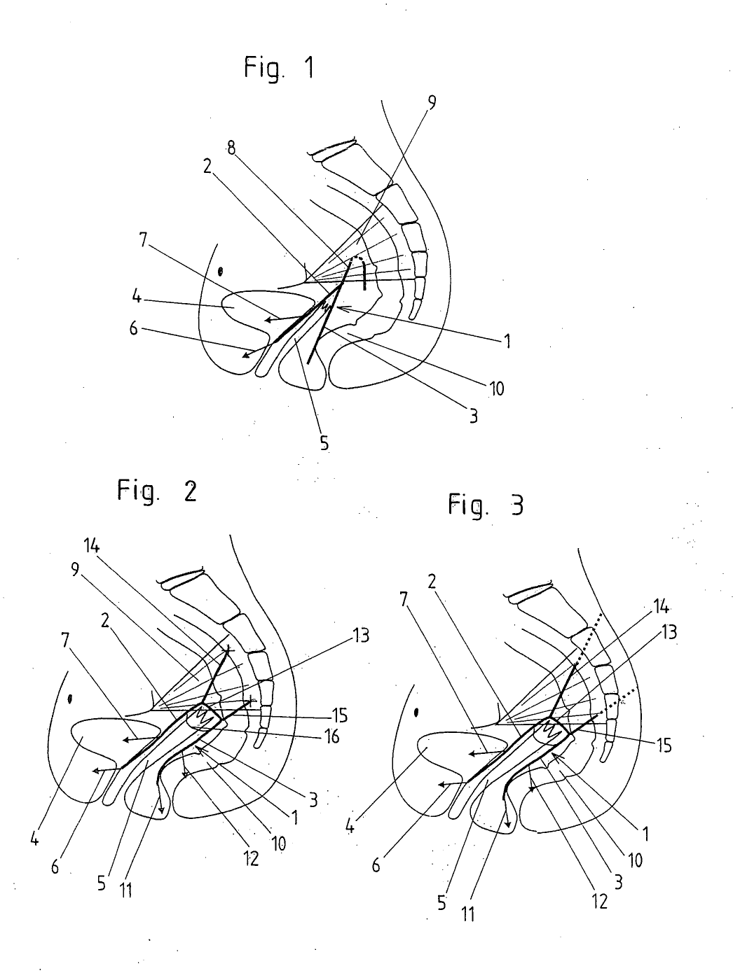

FIG. 1 depicts schematically a total surgical reconstruction in the area of the pelvic floor with an implantable mesh according to prior art. The mesh 1 comprises an anterior mesh segment 2 disposed between the bladder 4 and the vagina 5 and a posterior mesh segment 3 disposed between the vagina 5 and the rectum 10. The anterior mesh segment 2 is equipped with a pair of distal transobturator holding straps 6 and a pair of proximal transobturator holding straps 7, which, each starting from the anterior mesh segment 2, extend laterally and ventrally. These holding straps and their courses are only indicated schematically by arrows in FIG. 1. These holding straps 6, 7 are passed through the obturator in the implantation of the mesh.

The posterior mesh segment 3 is equipped with a pair of dorsal holding straps 8 which, starting from the proximal end of the posterior mesh segment 2, extend laterally and dorsally and are passed through the sacrospinous ligament 9. Thereby, seen in side view, overall an approximately V-shaped configuration of the anterior and posterior mesh segment 2, 3 results.

FIG. 2 shows highly schematically the positioning of a mesh according to the invention in a total surgical reconstruction in the area of the pelvic floor, the uterus having been removed. The mesh comprises again an anterior mesh segment 2 disposed between the bladder 4 and the vagina 5 and a posterior mesh segment 3 disposed between the vagina 5 and the rectum 10. From the anterior mesh segment 2 extends a pair of distal transobturator holding straps 6 as well as a pair of proximal transobturator holding straps 7 extending laterally and ventrally, respectively, and which are passed through the obturator. These transobturator holding straps 6, 7 are again only indicated by arrows.

From the posterior mesh segment 3 extend a pair of distal translevator holding straps 11 as well as a pair of proximal translevator holding straps 12. Starting from the posterior mesh segment 3, these straps extend laterally and ventrally and are passed through the levator muscle. These translevator holding straps 11, 12 are only indicated schematically by arrows in FIG. 2.

Between the anterior mesh segment 2 and the posterior mesh segment 3 the mesh comprises an intermediate segment 13. From the proximal end of the anterior mesh segment 2 bordering on the intermediate segment 13 extends a pair of upper dorsal holding straps 14. Starting from the anterior mesh segment 2, these straps extend laterally and dorsally and are sutured to the sacrospinous ligament 9. They could instead also be passed through the sacrospinous ligament 9, be deflected ventrally in their further course and be passed through the skin below the pubic bone wherein, after the operation, the portion projecting from the skin is trimmed.

From the proximal end of the posterior mesh segment 3 bordering on the intermediate segment 13 extends a pair of lower dorsal holding straps 15. Starting from the posterior mesh segment 3, these extend laterally and dorsally and are sutured to the sacrospinous ligament 9. They could instead also be passed through the sacrospinous ligament 9, subsequently be deflected ventrally in their further course and be passed through the skin below the pubic bone, wherein after the operation, the portion projecting from the skin is trimmed.

In a mesh 1 according to the invention, seen in side view, an approximately U-shaped configuration of the anterior mesh segment 2, intermediate segment 13 and posterior mesh segment 3, is formed after the operation as is evident in FIG. 2. The reconstruction in the area of the proximal vagina 5 is thereby significantly improved, further, through the translevator holding straps 11, 12 the securement of the posterior mesh segment 3 is significantly improved.

In a preferred embodiment variant of the invention on the intermediate segment 13 are disposed bilaterally projecting flaps 16, which are only indicated in FIG. 2 by a line and are placed onto the lateral walls of the proximal vagina whereby an improved support of this area of the vagina is attained against being pushed in by adjacent body structures.

FIG. 3 shows highly schematically a slightly modified installation of a mesh 1 according to the invention. The upper and lower dorsal holding straps 14, 15 are here passed laterally and dorsally through the coccygeal muscle or the sacrotuberous ligament (which are not shown in FIG. 3 for the sake of clarity) and are subsequently passed dorsally and through the skin, wherein after the operation the portion projecting from the skin is trimmed. With this securement results also a substantially U-shaped configuration of the anterior mesh segment 2, the intermediate segment 13 and posterior mesh segment 3.

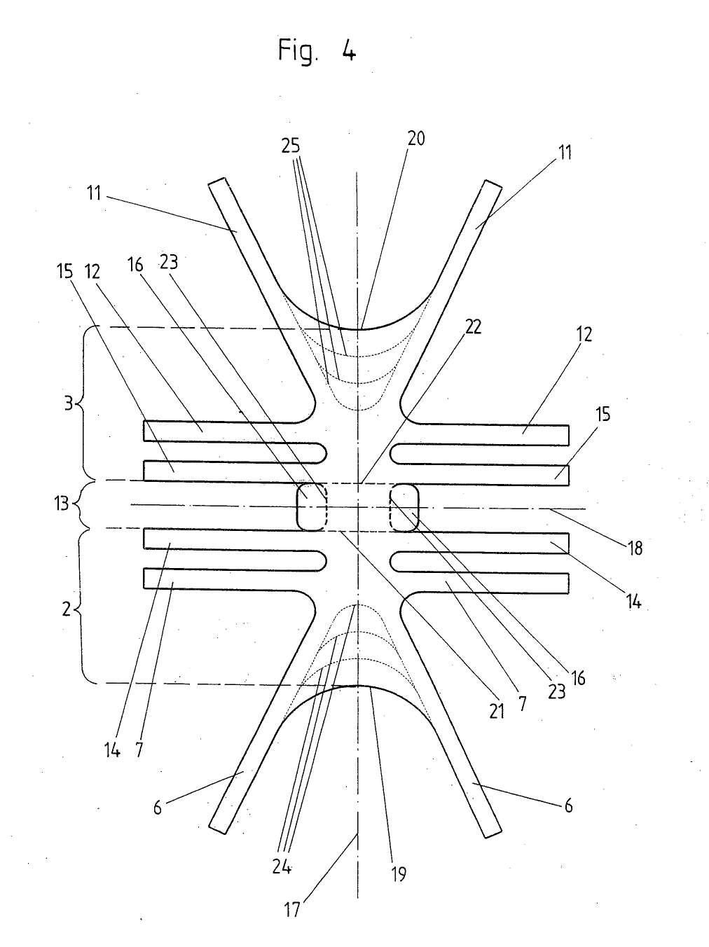

An advantageous embodiment variant of a mesh according to the invention is shown in greater detail in FIG. 4. The mesh is formed mirror-symmetrically with respect to a longitudinal center line 17, wherein the two holding straps 6, 7, 11, 12, 14, 15 of a particular pair are disposed mirror-symmetrically with respect to one another.

In the depicted embodiment variant the mesh is further formed mirror-symmetrically with respect to a transverse center line 18 centrally crossing the intermediate segment 13. A non-symmetric formation with respect to the transverse center line 18 is conceivable and feasible in order to permit further adaptation of the mesh to the body structures.

As already stated, from the anterior mesh segment 2 extend a pair of distal transobturator holding straps 6 and a pair of proximal transobturator holding straps 7. The distal transobturator holding straps 6 extend, for example, from the distal end 19 of the anterior mesh segment 2 at an angle between 20° to 60° to the longitudinal center line 17. The proximal transobturator holding straps 7 extend, for example, from a central region of the anterior mesh segment 2 at an angle of substantially 90° (i.e. discrepancies of ±20° should be within acceptable coverage) to the longitudinal center line 17.

From the anterior mesh segment 2 extends further a pair of upper dorsal holding straps 14 and specifically from that portion of the anterior mesh segment 2 which borders on the intermediate segment 13. For example, the upper dorsal holding straps 14 extend at an angle of substantially 90° (i.e. discrepancies of ±20° each should be within acceptable coverage) to the longitudinal center line 17.

From the posterior mesh segment 3 extends a pair of lower dorsal holding straps 15, and specifically from that portion of the posterior mesh segment 3 which borders on the intermediate segment 13. The lower dorsal holding straps 15 extend, for example, from the posterior mesh segment at an angle of substantially 90° (i.e. discrepancies of ±20° each should be within acceptable coverage) to the longitudinal center line 17.

From the posterior mesh segment furthermore extends preferably at least one pair of translevator holding straps 11, 12. These extend from a portion of the posterior mesh segment 3 which is located further at the distal end 20 of the posterior mesh segment 3. In the depicted embodiment example a pair of distal translevator holding straps 11 and a pair of proximal translevator holding straps 12 are provided. The distal translevator holding straps 11 extend from the distal end 20 of the posterior mesh segment 3, wherein, for example, they form an angle in the range between 20° to 60° with the longitudinal center line 17. The proximal translevator holding straps 12 extend from a central region of the posterior mesh segment 3, wherein they form, for example, an angle of substantially 90° (i.e. a discrepancy of ±20° is to be within acceptable coverage) with the longitudinal center line 17.

The intermediate segment 13 located between the sites from which extend the upper and lower dorsal holding straps 14, 15, has a substantially lesser extend in the direction of the longitudinal center line 17 than the anterior and the posterior mesh segment 2, 3. During the implantation of the mesh the intermediate segment 13 with respect to the anterior mesh segment 2 and the posterior mesh segment 3 with respect to the intermediate segment 13 is folded over or bent over in the same direction. Appropriate bending lines 21, 22 parallel to the transverse center line 18 are shown as dot-dash lines. In practice, folding-over at a certain radius will be more likely than a sharp bent-off. The folding-over between the anterior mesh segment 2 and the intermediate segment 13 as well as between the intermediate segment 13 and the posterior mesh segment 3 in each case is in the range of substantially 90° (i.e. discrepancies of ±20° each should be within acceptable coverage).

At the intermediate segment 13 flaps 16 projecting laterally on both sides are disposed. During the implantation these are folded over about bending lines 23 parallel to the longitudinal center line 17 to be brought into contact on the lateral walls of the proximal vagina, which bending lines are depicted in FIG. 4 by dotted lines. Again, in the implanted state, folding at a certain radius rather than a sharp bent will more likely result.

In the region of the distal ends 19, 20 of the anterior and posterior mesh segment 2, 3 markings 24, 25 are applied onto the mesh 1. Thereby intraoperative sizing of the mesh 1 in order to adapt the mesh 1 to the particular patient is facilitated. Such markings can also be applied, for example, in the region of the flaps 16.

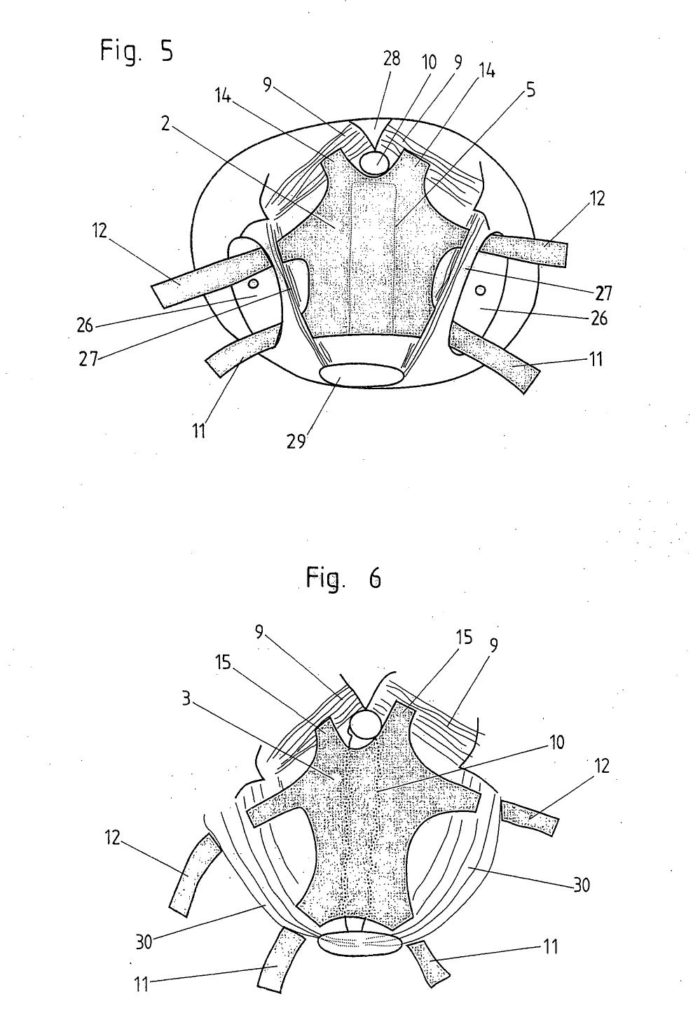

FIGS. 5 and 6 show schematic illustrations of the disposition of the anterior and posterior mesh segment 2, 3 in the implanted state of mesh 1 from a viewing angle differing from that of FIG. 2 (each in top view onto the corresponding mesh segment). Mesh segments 2, 3 are shown in the schematic illustrations of FIGS. 5 and 6 slightly differently compared to FIG. 4 (in particular differing in their ratios of length to width), however, the mesh segments 2, 3 can have the form depicted in FIG. 4.

In FIG. 5 is evident the manner in which the transobturator holding straps 6, 7 are passed through the particular obturator 26. They can be passed through the “tendinous arch of the pelvic fascia” 27. The upper dorsal holding straps 14 are sutured tight to the particular sacrospinous ligament 9. In FIG. 5, furthermore, the sacrum 28, the pubic bone 29, the rectum 10 and the vagina 5 (in dotted lines) located beneath the anterior mesh segment 2 are indicated.

In FIG. 6 the passing of the translevator holding straps 11, 12 through the particular levator muscle 30 is evident, the distal translevator holding straps 11 being passed further distally than the proximal translevator holding straps 12 through the particular levator muscle 30. In FIG. 6 further is indicated that the lower dorsal holding straps 15 are sutured to the particular sacrospinous ligament 9.

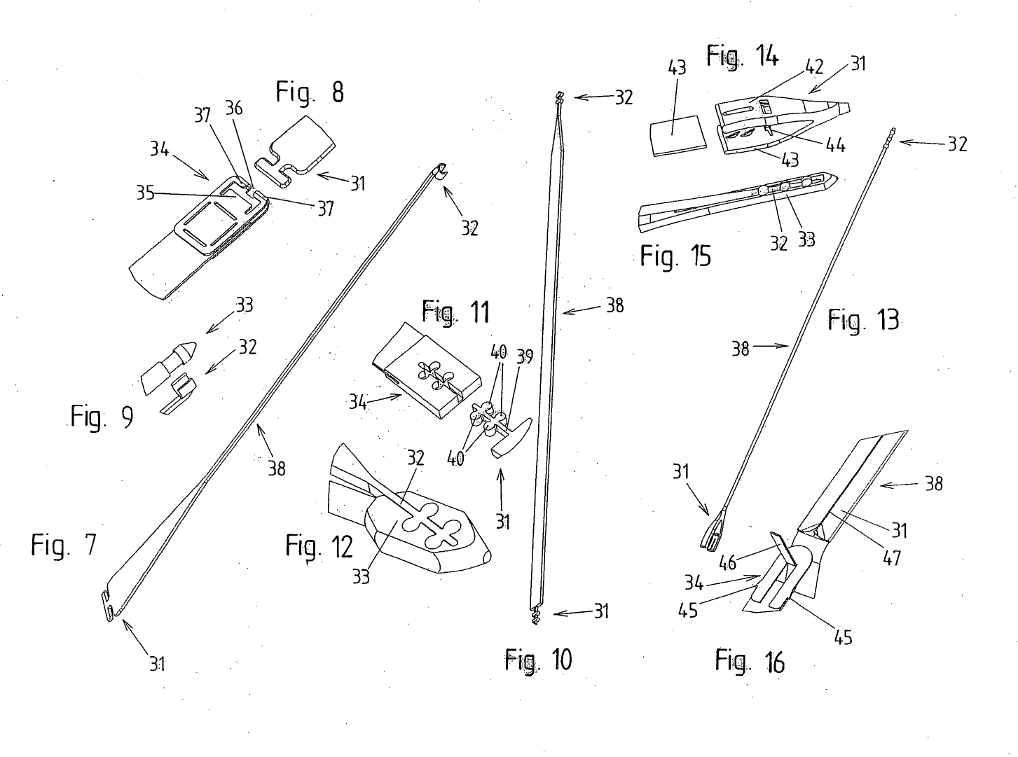

A medical instrument 38 for pulling a holding strap through a tissue channel is depicted in FIG. 7. The instrument 38 includes a T-shaped connection section 31 for hooking in the free end of the holding strap. The instrument 38 tapers toward the connection section 31 up to the width of the holding strap, the instrument having a small thickness. At the other end is provided a coupling part 32 for coupling on a further medical instrument, of which in FIG. 9 a coupling section 33 is shown.

The holding strap includes at the end a connection element 34 (cf. FIG. 8). In the depicted embodiment example this element is formed by a metal piece into which the T-shaped connection section 31 can be hooked. In the hooked-in state a spring-elastic tab 35 secures the connection section 31 in its hooked-in state. For hooking-in the connection section 31 the connection element 34 includes on both sides of an opening 36 U-shaped sections 37, into which the T-web of the connection section 31 can be hooked.

Instead of disposing a connection element 34 at the end of the holding strap, the end of the holding strap can also be turned over and sewn up whereby a loop is formed. Into this loop a slit could be introduced from the end such that on both sides of the slit suspension loops result into which the T-web of the connection section 31 can be hooked.

By means of such a medical instrument 38 a particular holding strap of the mesh 1 can be pulled into the tissue free of folds. In the implantation of the mesh preferably a number of instruments 38 corresponding to the number of holding straps is introduced into the body. The holding straps are subsequently coupled to the connection section 31 of the instruments 38 and following such, the holding straps are pulled through the tissue by means of the instruments 38.

A further embodiment variant of an instrument 38 for pulling in a holding strap is depicted in FIG. 10. The connection section 31 of instrument 38 includes here a forwardly projecting web 39 with lateral extensions 40 (cf. FIG. 11). The connection element 34 at the end of the holding strap is provided with an indentation, the form of which corresponds to the connection section 31 in order to make possible the coupling of the connection section 31 to the connection element 34.

The instrument 38 can again be coupled at the other end by means of a coupling part 32 to a coupling section 33 of a further medical instrument with which the instrument 38 can be manipulated. This coupling can, for example, be implemented in an analogous manner as the coupling to the holding strap (cf. FIG. 12).

The medical instrument 38 according to this embodiment variant, again, adjoining the connection section 31 has a shape substantially corresponding to the holding strap (in width and thickness), which is here continued over the major portion of the length of the instrument.

A further embodiment of a medical instrument 38 for pulling in a holding strap is depicted in FIG. 13. The connection section 31, shown at an enlarged scale in FIG. 14, is here formed in the shape of tongs. Between the two tong parts 41, 42 the end 43 of the holding strap can be inserted and subsequently fixed, by pressing the tong parts 41, 42 together and snapping them together in the pressed-together position by means of a snapping element 44. In this embodiment no special connection element needs to be disposed at the end of the holding strap.

At the other end of the instrument, again, a coupling part 32 for coupling to a coupling section 33 of a further medical instrument is provided (cf. FIG. 15).

FIG. 16 shows a further feasibility for connecting a holding strap with a medical instrument for pulling in the holding strap. The medical instrument 38 includes here, at least in the region of an end-side connection section 31, a forwardly open insertion channel formed in the shape of a flat rectangle. At the end of the holding strap is disposed a connection element 34. This element includes a flat metal piece with laterally projecting sawtooth-form points 45. After the connection element 34 has been introduced into the insertion channel of the connection section 31, these points 45 counteract the pulling-out of the connection element 34. The connection element 34 comprises further a spring-elastic tab 46 projecting from the plane of the flat metal part, which tab also serves for securing the connection element 34 in the connection section 31. If the connection element 34 is to be removed again from the connection section 31, the connection section 31 is torn open along a preset tear-open line 47 for this purpose.

The mesh is comprised of a histocompatible material into which body tissue can grow. The mesh is, for example, comprised of polypropylene.

As is evident in the preceding description, the scope of the invention is not limited to the depicted embodiment examples but rather, with reference to the attached claims, should be determined with its full range of feasible equivalents. While the preceding description and the drawing represent the invention, it is obvious to a person of skill in the art that various modifications can be carried out therein without leaving the true spirit and scope of the invention.

### LEGEND TO THE REFERENCE NUMBERS

1 mesh
2 anterior mesh segment
3 posterior mesh segment
4 bladder
5 vagina
6 distal transobturator holding strap
7 proximal transobturator holding strap
8 dorsal holding strap
9 sacrospinous ligament
10 rectum
11 distal translevator holding strap
12 proximal translevator holding strap
13 intermediate segment
14 upper dorsal holding strap
15 lower dorsal holding strap
16 flap
17 longitudinal center line
18 transverse center line
19 distal end
20 distal end
21 bending line
22 bending line
23 bending line
24 marking
25 marking
26 obturator
27 fascia
28 sacrum
29 pubic bone
30 levator muscle
31 connection section
32 coupling part
33 coupling section
34 connection element
35 tab
36 opening
37 section
38 instrument
39 web
40 extension
41 tong part
42 tong part
43 end
44 snapping element
45 point
46 tab
47 tear-open line

1. An Implantable mesh for surgical reconstruction in the area of the pelvic floors, comprising
an anterior mesh segment for the disposition between the bladder and the vagina,
a posterior mesh segment for the disposition between the vagina and the rectum,
an intermediate segment located between the anterior mesh segment and the posterior mesh segment,
a pair of distal transobturator holding straps extending from the anterior mesh segment for the positioning starting from the anterior mesh segment and extending laterally and ventrally through the obturator,
a pair of proximal transobturator holding straps extending from the anterior mesh segment for the positioning starting from the anterior mesh segment and extending laterally and ventrally through the obturator and,
a pair of lower dorsal holding straps extending from the posterior mesh segment in the region of the posterior mesh segment bordering on the intermediate segment for the positioning starting from the posterior mesh segment and extending laterally and dorsally, and
a pair of upper dorsal holding straps extending from the anterior mesh segment in the region of the anterior mesh segment bordering on the intermediate segment for the positioning starting from the anterior mesh segment and extending laterally and dorsally.

2. The implantable mesh as claimed in claim 1, wherein the mesh is implemented mirror-symmetrically with respect to a longitudinal center line centrally crossing the segments of the mesh.

3. The implantable mesh as claimed in claim 2, wherein the mesh is implemented mirror-symmetrically with respect to a transverse center line centrally crossing the intermediate segment, which transverse center line (18) is at right angles to the longitudinal center line.

4. The implantable mesh as claimed in claim 2, that wherein the distal transobturator holding straps extend from a distal end of the anterior mesh segment at an angle between 20° to 60° to the longitudinal center line.

5. The implantable mesh as claimed in claim 2, wherein the proximal transobturator holding straps extend from a central region of the anterior mesh segment at an angle of substantially 90° to the longitudinal center line.

6. The implantable mesh as claimed in claim 2, wherein the upper dorsal holding strap extends from the anterior mesh segment an angle of substantially 90° to the longitudinal center line.

7. The implantable mesh as claimed in claim 2, wherein the lower dorsal holding strap extends from the posterior mesh segment at an angle of substantially 90° to the longitudinal center line.

8. The implantable mesh as claimed in claim 1, wherein from the posterior mesh segment, further, extends at least one pair of translevator holding straps for the positioning starting from the posterior mesh segment and extending laterally and ventrally through the levator muscle.

9. The implantable mesh as claimed in claim 8, wherein from the posterior mesh segment extend a pair of distal translevator holding straps and a pair of proximal translevator holding straps.

10. The implantable mesh as claimed in claim 9, wherein the distal translevator holding straps extend from a distal end of the posterior mesh segment at an angle between 20° to 60° to the longitudinal center line.

11. The implantable mesh as claimed in claim 8, wherein the proximal translevator holding straps extend from a central region of the posterior mesh segment at an angle of substantially 90° to the longitudinal center line.

12. The implantable mesh as claimed in claim 1, wherein the intermediate segment comprises bilaterally projecting flaps for the placement on lateral walls of the proximal vagina.
